(19) United States
(12) Patent Application Publication
(10) Pub. No.: US 2023/0218446 A1
(43) Pub. Date: Jul. 13, 2023

(54) ADHESIVE TRANSPARENT MULTIFUNCTIONAL WOUND-COVERING MATERIAL AND MANUFACTURING METHOD THEREOF

(71) Applicant: SU-BIO CO.,LTD., Busan (KR)

(72) Inventor: I SU KIM, Busan (KR)

(21) Appl. No.: 18/009,020

(22) PCT Filed: Jul. 4, 2021
(86) PCT No.: PCT/KR2021/008466

§ 371 Date: Dec. 8, 2022

(30) Foreign Application Priority Data
May 3, 2021 (KR) 10-2021-0057258

(51) Int. Cl. A61F 13/02; A61L 15/24; A61L 15/44

(52) U.S. Cl. CPC A61F 13/0213; A61L 15/24; A61L 15/44; A61F 2013/00182

(57) ABSTRACT

The present invention has absorbency for the exudate of the wound surface and can maintain a moist environment suitable for promoting healing of the wound surface for a long time, an adhesive transparent multi-functional wound-covering material and a method for manufacturing the same are proposed so as not to cause pain or damage to the regenerated skin when replacing the wound dressing. Such a wound-covering material includes 14% by weight of polyvinylpyrrolidone, 5% by weight of propylene glycol, 79.0996% by weight of water, 0.6% by weight of calcium chloride, 0.3% by weight of potassium sorbate, 0.0004% by weight of RH oligopeptide, and 1% by weight of D-xylitol.

### TECHNICAL FIELD

The present invention relates to an adhesive transparent multifunctional wound-covering material and a method for manufacturing thereof, and more specifically, it has excellent elasticity and absorbency of the exudate on the wound surface and can maintain a moist environment suitable for promoting the healing of the wound surface for a long time. In addition, the present invention relates to an adhesive transparent multifunctional wound-covering material that does not worry about pain or damage to the regenerated skin when changing the wound dressing, and a manufacturing method thereof.

### BACKGROUND TECHNOLOGY

The skin of the human body has the property of defending the wound area and healing it naturally in the event of wounds and burns, and in this case, wound covering materials are used as a way to effectively protect the wound area and speed up healing.

As the wound dressing, gauze, powder, spray, ointment, cream, sponge, and the like have been used.

Most of these were intended to heal by drying the wound area by absorbing the exudate from the wound surface.

According to a 1962 study by zoologist Winter, it was disclosed that keeping the wet dressing is more helpful for healing than drying the wound to produce scabs, and the usefulness of wet dressing has been continuously proven and emphasized since the Winter paper. These days, wet dressing, which prevents bodily fluid secreted from the wound from dehydration or drying, is widely recognized as facilitating wound healing.

Therefore, the characteristics of the wound covering material should be that it can effectively absorb blood and exudate generated from wounds such as wounds and burns, maintain a moist environment, and at the same time effectively protect the wound area.

In addition, the fact that there should be no rejection reaction to the wound site due to excellent biosynthesis, and that it should be moisture permeable to maintain high moisture permeability to prevent maceration of normal skin around the wound is also a characteristic required for wound dressing.

There is a problem that moisture permeability drops rapidly as the thickness of the wound coating becomes thicker. Therefore, if the film is thinly processed to increase the moisture permeability, the moisture permeability increases, but if the film becomes too thin, there is a problem that it is difficult to handle when treating the wound.

Non-woven fabrics and paper used as wound dressings have low prices and are easy to use, but they do not have a protective function or waterproof properties against bacteria or foreign substances, and there is a problem that they fall off easily when applied to curved parts of the body.

In addition, since the moisture permeability is too high to keep the wound dry, the absorbent material is attached to the wound surface, and there is a problem that it causes damage to the new tissue when the wound-covering material is exchanged.

### CITED REFERENCES

Patent Document 0001: Korea Patent Publication No. 10-2010-0126297 (Hydrogel wound covering, published date: Dec. 1, 2010).
Patent Document 0002: Korea Patent Publication No. 10-2015-0088652 (Hydration Gel-type Silk Fibroin wound coating and its manufacturing method, published date: Aug. 3, 2015).
Patent Document 0003: Korea Patent Publication No. 10-2020-0020618 (Method of manufacturing wound cladding composition with improved cohesiveness, published date: Feb. 26, 2020).
Patent Document 0004: Korean Patent Publication No. 10-2271980 (Collagen-Alginate wound coating and its manufacturing method, published date: 07/02/2021).

### DESCRIPTION OF THE INVENTION

### Technical Problems to be Solved

The present invention has excellent elasticity, has absorbency for the extract of the wound surface, can maintain a moist environment suitable for promoting healing of the wound surface for a long time, and does not cause pain or damage to the regenerated skin when exchanging the wound dressing material. The purpose is to provide an adhesive transparent multifunctional wound dressing and a method for manufacturing the same.

### Technical Solutions

The adhesive transparent multifunctional wound-covering material according to the present invention may include 14% by weight of polyvinylpyrrolidone, 5% by weight of propylene glycol, 79.0996% by weight of water, 0.6% by weight of calcium chloride, 0.3% by weight of potassium sorbate, 0.0004% by weight of RH oligopeptide and 1% by weight of D-xylitol.

### Effects of the Invention

The adhesive transparent multifunctional wound covering material according to the present invention has excellent protection of the wound surface and the absorbency of the exudate, so that a moist environment suitable for promoting the healing of the wound surface can be maintained for a long time, and also when the wound coating is exchanged, it has the advantage of not damaging regenerated skin and no pain.

### EMBODIMENTS OF THE INVENTION

Hereinafter, embodiments according to the present invention will be described in detail with reference to the accompanying drawings.

Detailed descriptions of the present invention will be made with reference to the accompanying drawings illustrating specific embodiments of the present invention as examples. It should be understood that various embodiments of the present invention are different but are not necessarily mutually exclusive. For example, a specific shape, structure, and characteristic of an embodiment described herein may be implemented in another embodiment without departing from the scope and spirit of the present invention

In the present invention, Polyvinylpyrrolidone is used as the main raw material in the composition of the wound dressing, and the wound covering material is applied to the wound area in a form of a film to form a protective film to realize an adhesive transparent multifunctional wound covering material that can protect the wound from external factors.

Preferably, the wound covering of the present invention consists of 14% by weight of polyvinylpyrrolidone, 5% by weight of propylene glycol, 79.0996% by weight of water, 0.6% by weight of calcium chloride, 0.3% by weight of potassium sorbate, 0.0004% by weight of RH oligopeptide and 1% by weight of D-xylitol.

In addition, a method of preparing a wound-covering material according to the present invention may include:

drying a conventional agitator, adding about 10,000 ml of water as a solvent to the agitator, and then adding 14% by weight of polyvinylpyrrolidone to dissolve while heating the agitator, and when polyvinylpyrrolidone is dissolved in the step, 0.6% by weight of calcium chloride is added to the agitator and rotated, and then the heating of the agitator is stopped, and 0.3% by weight of potassium sorbate, 0.0004% by weight of RH oligopeptide and 1% by weight of D-xylitol are added to the agitator, and stirring agitator for producing wound-covering material.

### DETAILED EMBODIMENTS OF THE INVENTION

Hereinafter, a preferred embodiment of the present invention will be described in detail as follows. In the detailed description to be described below, representative embodiments of the present invention will be presented to achieve the above-described technical problems. And other embodiments that may be presented with the present invention are replaced by descriptions in the configuration of the present invention.

The adhesive transparent multifunctional wound dressing material according to a preferred embodiment of the present invention may include 14% by weight of polyvinylpyrrolidone, 5% by weight of propylene glycol, 79.0996% by weight of water, 0.6% by weight of calcium chloride, 0.3% by weight of potassium sorbate, 0.0004% by weight of RH oligopeptide and 1% by weight of D-xylitol, which is specifically described in Table 1 below.

TABLE 1

| Product | Raw material or | Weight | Purpose of | Volume |
|---|---|---|---|---|
| Name | ingredient name | (%) | addition | (ml) |
| Wound | Polyvinylpyrrolidone | 14 | Film formation | 2800 |
| dressings | Propylene Glycol | 5 | Emulsifiers | 1000 |
| (Mu-H10) | water | 79.0996 | Solvent action | 15819.92 |
| | calcium chloride | 0.6 | Increase | 120 |
| | | | viscosity | |
| | Potassium sorbate | 0.3 | Preservatives | 60 |
| | IndiLipo rhEGF | 0.0004 | Moisturizer | 0.08 |
| | D-Xylitol | 1 | sweetener | 200 |

As shown in Table 1 above, Polyvinylpyrrolidone was used as artificial plasma in Germany during World War II, which began in 1939, and attracted attention from countries after the war.

Previously called Haemodyn. In addition, the substitute plasma manufactured by Farbenfabriken Bayer A.G. (FBy) in Germany is called Peristone, and it is obtained by dissolving 3.5% of polyvinylpyrrolidone (average molecular weight: 25,000) in dilute saline.

In order to realize the efficacy of such polyvinylpyrrolidone in the wound dressing of the present invention, the present inventor(s) have examined it in the present invention, and found that a wound dressing material is prepared with a composition consisting of a hydrogel composed of polyvinylpyrrolidone, the main raw material for water-soluble synthetic or semi-synthetic polymers, contained in water and RH Oligopeptides(IndiLipo rhEGF), an epithelial cell growth factor and the wound dressing material to the applied to a wound to form a protective film in the form of a film to protect the wound.

In the present invention, propylene glycol is used as an emulsifier, wherein propylene glycol has the formula C₃H₈O₂, specific gravity is 1.036 to 1.040, boiling point is 185 to 189° C., can be mixed with water, alcohol, acetone, ethyl acetate, chloroform, ether, etc., dissolves gasoline and is not mixed with petroleum ether and paraffin, and is hygroscopic but not volatile.

In the present invention, propylene glycol is used as an emulsifier, wherein propylene glycol has the formula C₃H₈O₂, specific gravity is 1.036 to 1.040, boiling point is 185 to 189° C., mixed with water, alcohol, acetone, ethyl acetate, chloroform, ether, etc., dissolves gasoline and is not mixed with petroleum ether and paraffin, and is hygroscopic but not volatile.

In addition, it is stable to heat and sunlight, but flammable (flash point 104° C.), and has excellent solubility compared to glycerin, so it was used as a substitute for glycerin shortages during World War II.

Meanwhile, in the present invention, water is used as a solvent, and calcium chloride is used for the purpose of increasing the viscosity of the wound dressing.

In other words, the wound covering of the present invention requires adhesion enough to follow the movement of the skin when applied to the wound, and adhesive force enough not to damage the regenerated skin during exchange, and is added for the purpose of satisfying it.

The present invention uses potassium sorbate as a preservative, which potassium sorbate is used to inhibit mold and yeast in many foods such as cheese, wine, yogurt, and bakery products, and can also be found in the ingredient list of many dried fruit products.

In addition, plant-based food supplements generally contain potassium sorbic acid, which prevents mold and microorganisms, prolongs the storage period, and is used only in small amounts, so it does not adversely affect health.

And it is used in many health foods to inhibit the growth of microorganisms to increase the shelf life, and some manufacturers use the preservatives instead of parabens.

In addition, the RH oligopeptide (IndiLipo rhEGF) used as a composition of the wound-covering material in the present invention is a water-soluble peptide drug, in particular rhEGF (epithelial growth factor) is used as a moisturizer in the wound dressings according to aspect(s) of the present invention. And D-xylitol is a pentitol (five-valent alcohol) type natural sugar alcohol, and is used as a sweetener according to aspect(s) of the present invention.

The following describes in detail a method of producing an adhesive transparent multifunctional wound coating material of the present invention composed of the above composition.

First, stirring until there are no foreign substances in the agitator, washing with a conventional emulsifying device, and then heating the cleaned agitator to dry the agitator. At this time, the heating temperature for drying the agitator is preferably set to about 75°.

Thereafter, after adding about 10,000 ml of water, which is a solvent, to the dried agitator, the agitator is started to be heated, and at this time, whenever the temperature of the water is 35° C. or higher, and polyvinylpyrrolidone is slowly poured into the agitator little by little while the emulsifier is rotated about 20 times.

In other words, if you pour polyvinylpyrrolidone all at once, it is easy to form lumps, so if you visually check that polyvinylpyrrolidone is dissolved in water, pour it gradually.

Next, dissolve about ½ of polyvinylpyrrolidone dissolved in water with an emulsifier, then add 10,000 ml of water to the agitator again and turn the agitator again. Thereafter, whenever the temperature of the water reaches 35° C., the remaining polyvinylpyrrolidone is poured into the agitator a total of three times. At this time, be careful that the solution may splash in the process of pouring the polyvinylpyrrolidone and pour all 2,800 ml of polyvinylpyrrolidone into an agitator and dissolve it in water.

Thereafter, after putting the lid on the agitator, rotate the agitator for about 30 minutes so that Polyvinylpyrrolidone is completely dissolved in water, stop the rotation of the agitator, open the lid, add 120 ml of calcium chloride to the agitator, and then rotate the agitator again for 1 hour, and then stop the heating of the agitator, and then the remaining ingredients, potassium sorbate 0.3% by weight, RH oligopeptide 0.0004% by weight and 1% by weight of D-xylitol is added, and then the agitator and emulsifier are stirred and emulsified for 2 hours and 30 minutes to prepare an adhesive transparent multifunctional wound-covering material.

The above description is merely an illustrative description of the present invention, and various modifications will be possible by those skilled in the art to which the present invention belongs, to the extent that it does not depart from the technical spirit of the present invention.

Therefore, the embodiments disclosed in the present invention are not limited to the present invention. The scope of the present invention should be construed according to the following claims, and all techniques within the equivalent range should be construed as being included in the scope of the present invention.

What is claimed is:

1. An adhesive transparent multifunctional wound-covering material, comprising:
14% by weight of polyvinylpyrrolidone;
5% by weight of propylene glycol;
79.0996% by weight of water;
0.6% by weight of calcium chloride;
0.3% by weight of potassium sorbate;
0.0004% by weight of RH oligopeptide; and
1% by weight of D-xylitol.

2. A method of producing an adhesive transparent multifunctional wound-covering material, the method comprising:
Inputting about 10,000 ml of water as a solvent to an agitator after drying the agitator;
adding 14% by weight polyvinylpyrrolidone to the agitator to dissolve while heating the agitator;
adding 0.6% by weight of calcium chloride to the agitator and rotating when polyvinylpyrrolidone is dissolved in the above step;
stopping the agitator, and then adding 0.3% by weight of potassium sorbate, 0.0004% by weight of RH oligopeptide and 1% by weight of D-xylitol; and
agitating the agitator for producing the adhesive transparent multifunctional wound-covering material.
